(19) United States
(12) Patent Application Publication
(10) Pub. No.: US 2024/0156636 A1
(43) Pub. Date: May 16, 2024

(54) FORCED AIR WARMING UNIT CONNECTION PORT CONTROLLER

(71) Applicant: 3M INNOVATIVE PROPERTIES COMPANY, St. Paul, MN (US)

(72) Inventors: Michael J. Woodson, West St. Paul, MN (US); Jared J. Balthazor, Linwood TWP, MN (US); Philip G. Dion, Blaine, MN (US); Jenna L. Lindsay, Woodbury, MN (US); Leroy J. Longworth, Woodbury, MN (US); Melissa T. Nelson, Woodbury, MN (US); Tyler J. Sandback, Prior Lake, MN (US); Michele A. Waldner, Hudson, WI (US); Jeffrey G. Zinn, Roseville, MN (US)

(21) Appl. No.: 18/282,110

(22) PCT Filed: Mar. 2, 2022
(86) PCT No.: PCT/IB2022/051843

§ 371 Date: Sep. 14, 2023

Related U.S. Application Data
(60) Provisional application No. 63/200,725, filed on Mar. 24, 2021.

(51) Int. Cl. A61F 7/00; A61M 39/10

(52) U.S. Cl. CPC A61F 7/0085; A61F 7/0097; A61M 39/1011; A61F 2007/006; A61F 2007/0094; A61F 2007/0095

(57) ABSTRACT

A method of manipulating a quick connect/disconnect system includes connecting a first coupling portion of a first connecting member to a fluid conductor and inserting a second coupling portion of the first connecting member within a third coupling portion of a second connecting member. The method further includes inserting the third coupling portion within a ring assembly which is rotatably coupled to the first coupling portion and engaging a mating feature on an external surface of the third coupling portion with a mating feature on an internal surface of the ring assembly. The method also includes rotating the ring assembly about the third coupling portion while the first mating portion is engaged with the second mating portion; and forcing the second coupling portion to move further within the third coupling portion by rotation of the ring assembly.

### TECHNICAL FIELD

The present invention relates generally to thermal comfort. More particularly, the present invention relates to a portable warming apparatus for providing pressurized, thermally conditioned air to a pneumatic, convective device.

### BACKGROUND

The human body requires a controlled temperature for normal physiological processes to work properly. Because of this need, the body is endowed with a complex array of systems for regulating thermal homeostasis. The heat of metabolism is substantial and must be exhausted to the surrounding environment to prevent overheating. However if the heat loss is too great, hypothermia may result. For this and other reasons the body is equipped to maintain thermal equilibrium for a broad range of environmental conditions. To aid the body in maintaining homeostasis and prevent hypothermia, patients are often provided a warming garment connected to a forced air warming unit which provides conditioned air to the garment covering the patient.

The forced air warming device may be configured to supply air at various temperatures and air pressures. The forced air warming device may include an interface for controlling various functions of the forced air warming device. Since warm air originates from the forced air warming unit, the interface is located on the forced air warming unit and generally out of reach of the patient. As such, the patient is unable to adjust the function of the forced air warming unit without the aid of a technician.

### SUMMARY

The current industry state of connecting hose and hose-end wires for a forced air warming device is a cumbersome and multi-step operation that is not recommended to be performed by a technician outside of servicing. As such, current forced air warming systems are unable to be replaced and interchanged with multiple hoses or warming garments connected to forced air warming devices. Further, when hose-end accessories, sensors, or controls are present and pass through the hose it may not be possible to perform a hose replacement without disassembly of the device. the warming devices require operation and control via the warming device, which prevents the patient from having access to changes the operation of the warming unit.

There may be substantial variation in person-to-person preferences in heat consumption. Some patients may require more or less heat than standard heating conditions supplied by previous forced air warming devices. For instance, certain patients may require increased temperatures due to the patient or operating environment. As such, control of the microenvironment must be given to the patient, for only the patient knows the specific point at which thermal comfort is provided. As such, it is important to provide a control interface that is accessible to an immobile patient that may be confined to a hospital bed or any other condition that may limit mobility of the patient.

There are many possible ways to warm a patient in a clinical setting. If the intent is to treat or prevent hypothermia, little attention needs to be paid to subjective feeling of the patient—the problem is merely an exercise in heat transfer theory. However, if the needs of thermal comfort are considered, as is the case with the patient warming system of the current disclosure, the problem becomes much more stringent. In this case, the target temperature range of the microenvironment is then critical. Also, the heating mechanism must be reasonably uniform over the body. Any large temperature variations are likely to result in discomfort.

The current forced air warming system provides a seamless and secure connection of the hose via a rotatable cam that provides the user with increased flexibility of hose assembly and removal. The cam includes at least one groove for receiving and retaining a tab located on an outer coupling of a forced air warming device. The cam is rotatable such that the tab is retained in the groove and an air-tight seal is formed between the hose and the outer coupling of the forced air warming device.

The outer coupling includes a first electrical coupling configured to be coupled to a second electrical coupling located on a distal end of the hose. The first electrical coupling includes a plurality of electrical components in communication with a control processor for controlling the operation of the forced air warming device. The second electrical coupling includes a plurality of electrical components in communication with a control interface located on a proximal end of the hose near the patient warming device. Coupling of the first electrical coupling to the second electrical coupling provides a communication interface between the control interface of the hose and the control processor disposed within the forced air warming device. Once the electrical couplings are secured, rotation of the cam in a first direction retains the tab of the outer coupling to the groove of the cam in order to ensure the electrical couplings remain in communication. The cam may then be rotated in a second direction to release the tab from the groove, and the hose may easily be removed from the outer coupling, providing easy servicing of the forced air warming device to various patient warming units and associated hoses.

The hose further includes a first wiring extending from the first electrical coupling near the distal end of the hose to the proximal end of the hose. The first wiring enables electronic communication between the second electrical coupling and the control interface. Further, the first wiring enables the control interface of the forced air warming device to be located near to the patient wearing the patient warming unit. Thus, the patient is able to control various operational functions of the forced air warming device in addition to a technician or a nurse.

### BRIEF DESCRIPTION OF DRAWINGS

FIG. 1 shows a forced air warming system including a forced air warming device and a patient warming unit in a clinical setting;

FIG. 2 illustrates a proximal end of a hose including an interface for controlling function of a forced air warming device;

FIG. 3 illustrates the forced air warming unit including an outlet for supplying pressurized air;

FIG. 4A illustrates a distal end of the hose including a rotatable cam for coupling to the forced air warming unit;

FIG. 4B illustrates a cross-sectional view of the distal end of the hose;

FIG. 5 illustrates the hose coupled to the outlet port of the forced air warming unit;

FIG. 6 illustrates a cross-sectional view of the hose coupled to the outlet port of the forced air warming unit.

### DETAILED DESCRIPTION

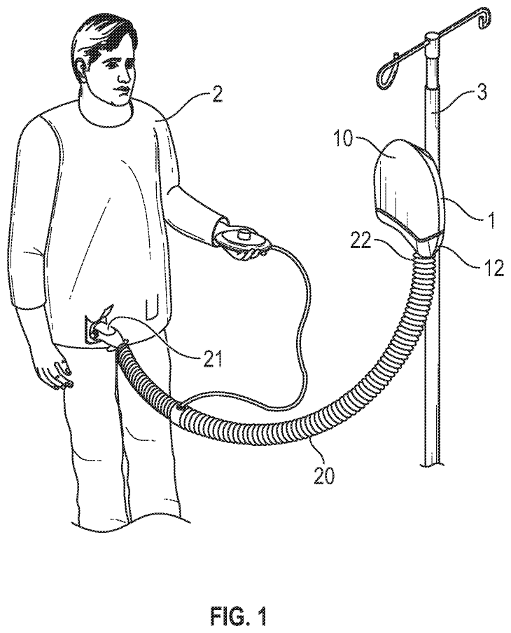

FIG. 1 illustrates a forced air warming device 1 and a patient warming unit 2. The forced air warming device 1 includes a device body 10 and an air outlet port 12 on the device body 10 10. A hose 20 connects and supplies warm air from the forced air warming device 1 to the patient warming unit 2. The hose 20 includes a proximal end 21 coupled to the patient warming unit 2 and a distal end 22 opposite the proximal end 21, the distal end 22 is coupled to the forced air warming device 1 at an outer coupling (not shown) proximate the air outlet port 12. In operation, the forced air warming device 1 produces a steam of pressurized, heated air which exits the device body 10 at the outlet port 12 and into the distal end 22 of the hose 20. The stream of pressurized, heated air is conducted by the hose 20 to the patient warming unit 2. The patient warming unit 2 receives the stream of pressurized air and emits the air through one or more surfaces for convective transfer of heat between the thermally conditioned air and the body of a patient. The forced air warming unit may have provisions for mounting on an IV pole 3.

Although the patient warming unit 2 is shown as a garment or hospital gown, the patient warming unit 2 may alternatively be a blanket, shirt or any other wearable garment capable of covering a portion of a patient's body and providing heated air to the patient.

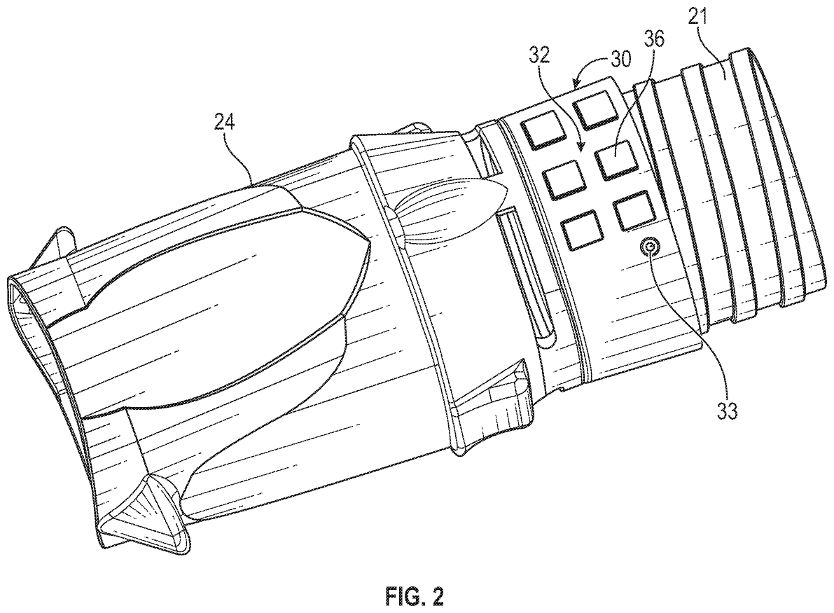

Referring to FIGS. 1 and 2, the proximal end 21 of the hose 20 includes a warming unit coupling 24. The warming unit coupling 24 includes a control interface 30 having a plurality of control feature 32 for controlling the function of the forced air warming device 1. The control features 32 may include a power interface 33 for switching the forced air warming device 1 on or off, adjusting the temperature of the forced air, adjusting the airflow, and any available function of the forced air warming device 1. The control interface 30 is located at the proximal end 21 of the hose 20 to allow for quick startup of the patient warming unit 2 and reduced workflow steps. The location of the control interface 30 enables a patient to have complete control of the operations of the forced air warming device 1 at a single control interface 30 while the patient is wearing the patient warming device 2. The location of the control interface 30 on the proximal end 21 of the hose 20 near the patient warming unit 2 provides the patient with easy access to the controls of the forced air warming device 1. The capability of warming unit coupling 24 of the hose 20 to be easily coupled and uncoupled to the patient warming device 1 provides greater flexibility in patient warming unit 2 design and interchangeability, while also enabling more accessible operation and an indication of the warming unit mode available to the patient wearing the patient warming unit 2.

The location of the control interface 30 enables a patient or subject wearing the patient warming unit 2 easy access to the control interface 30 in order to control the functioning of the forced air warming device 1. The control interface 30 may include an actuator/power switch 33 for controlling power of the forced air warming device 1. The control interface 30 may include the plurality of control feature 32 for controlling function and operation of the forced air warming device 1. The control features 32 of the interface may include options for increasing or decreasing the pressurization of the forced air, temperature of the forced air, timing of air supply, and any other features necessary for controlling air flow from the forced air warming device 1. The control interface 30 may additionally include a feedback indicator 36 for indicating the power status, mode of operation, operating condition and/or error status of the forced air warming device 1. The feedback interface 36 may include a plurality of LED lights or other features capable of indicating a status of the forced air warming device 1.

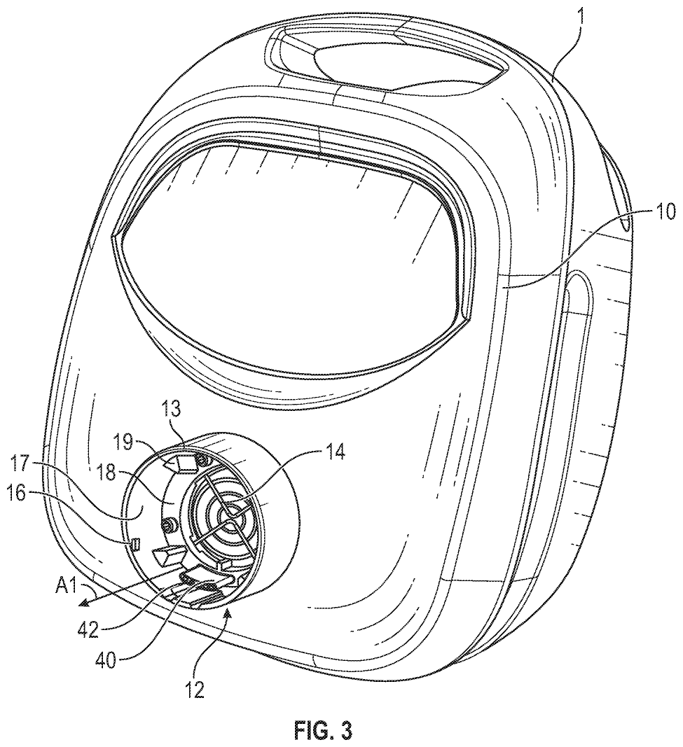

Referring to FIG. 3, the forced air warming device 1 is shown, including a device body 10 and an air outlet port 12 on the housing for supplying forced air from the forced air warming device 1 to the hose (not shown). The outlet port 12 includes an opening 13 and the opening 13 may include a grated filter 14 at the opening 13 to prevent inflow of unwanted debris into the forced air warming device 1. The forced air warming device 1 may include a heater plate (not shown) within the device body 10 near the outlet port 12. The heater plate is responsible for heating the forced pressurized air as it exits the forced air warming device 1. Additionally, the forced air warming device 1 may include an internal impeller (not shown) which rotates clockwise or counterclockwise to force air from the device body 10 and out of the outlet port 12 to the hose (not shown). The impeller pressurizes and forces the air from the outlet through the distal end of the hose to the proximal end of the hose where it enters the patient warming unit (not shown). The flow of pressurized air is indicated by arrow A1. As air is pressurized and flows through the device body 10, current is supplied to the heater plate, raising the temperature of the heater plate and thereby the temperature of the pressurized air as it is forced through the body to the outlet port 12.

The air outlet port 12 includes an outer coupling 13 for receiving and retaining the hose 20 to the forced air warming device 1. The outer coupling 13 further includes a wall 15 defining a periphery of the outer coupling 13 and at least one tab 16 extending inwardly from an inner surface of the wall 17. An outlet flange 18 may extend inwardly from the inner surface of the wall 17 configured to provide a stop point when the hose 20 is retained to the outlet port 12. A gasket 19 may be located on a surface of the flange 18. The gasket 19 provides an air-tight seal when the hose 20 is retained to the outlet port 12.

A first electrical coupling 40 is positioned within a circumference of the wall 17 of the outer coupling 13. The first electrical coupling 40 includes an electrical coupling tab 16 having a plurality of electrical components 42 that are in electrical communication with a control processor (not shown) of the forced air warming device 1. The electrical coupling tab 16 may extend outwardly from the surface of the outlet flange 18. The control processor is responsible for controlling operation of the forced air warming device 1. As will be discussed in greater detail below, the first electrical coupling 40 is configured to be electronically coupled to a second electrical coupling (not shown) of the hose 20. Because the hose 20 is removably coupled to the forced air warming device 1, it is necessary for the first electrical coupling 40 to be able to accommodate various electrical couplings from various hoses 20. By including an electrical coupling on the outer coupling 13 of the forced air warming device 1, the hose 20 may include electrical components extending the length of the hose 20 that are in communication with a control interface located on a proximal end of the hose (not shown).

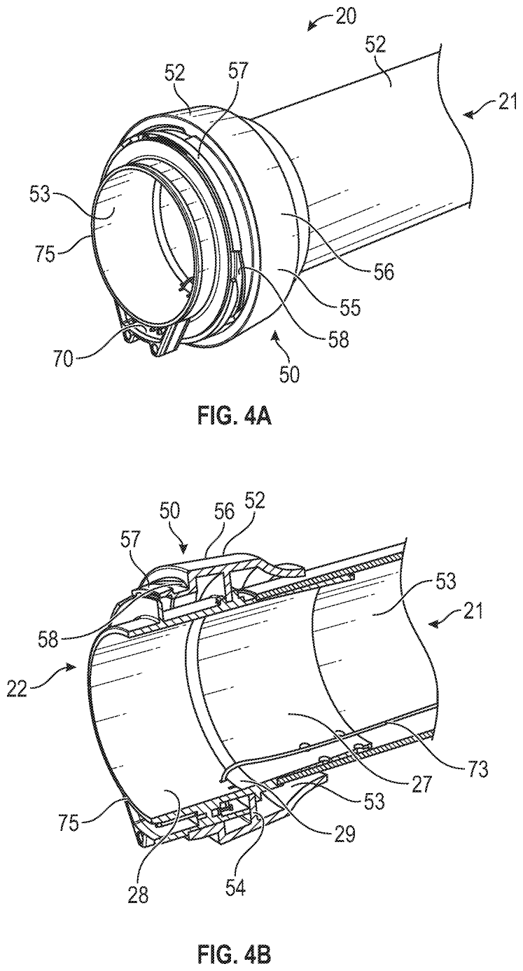

Referring to FIGS. 4B and 4B, a hose 20 for connecting to the forced air warming device 1 is shown. The hose 20 includes an outer surface 52 and an inner surface 53 (FIG. 4B) opposite the outer surface 52. The hose 20 additionally includes a proximal end 21 and a distal end 22 opposite the proximal end 21. The distal end 22 of the hose 20 is configured to be received and retained by the forced air warming device 1. The proximal end 21 of the hose 20 is connected to the patient warming unit 2 or garment. The hose 20 includes a first portion 27 of the hose 20 and a second portion 28 of the hose 20. The first and second portion 27, 28 of the hose 20 may be sonically welded to one another at a weld point 29. The second portion of the hose 20 includes the distal end of the hose 20 and the front edge 75.

A cam 50 is rotationally secured to the distal end 22 of the hose 20, the cam 50 including an outer surface 52 and an inner surface 53 opposite the outer surface 52. A rim 54 projects from the inner surface 53 of the cam 50 and is secured to the weld point 29 of the hose 20 between the first and second portion of the hose 20. The rim 54 is rotationally coupled to the hose 20 to allow rotation of the cam 50 in a clockwise or counterclockwise direction. The hose 20 includes a protuberance 55 extending around a circumference of the hose 20. The protuberance 55 provides a surface 56 for a user to grasp and rotate the cam 50 when coupling the hose 20 to the forced air warming device 1. A front lip 57 extends from the protuberance 55 towards the distal end 22 of the hose 20. The front lip 57 includes at least one groove 58 configured to accommodate the tab 16 that extends inwardly from the wall 17 of the outer coupling 13 of the forced air warming device 1 when the hose 20 is coupled to the forced air warming device 1. The at least one groove 58 may include a groove opening 59 configured to receive the tab 16 of the outer coupling 13. As the cam 50 is rotated, the tab 16 of the forced air warming device 1 is configured to slide away from the distal end 22 the hose 20 towards the proximal end 21 (as shown by arrow A2). As the tab 16 slides within the groove 58, the hose 20 is pulled inward toward the opening 13 of the outlet port 12 wherein a front edge of the hose 20 at the distal end 22 is pulled into contact with the flange 18 of the forced air warming device 1. In embodiments wherein the gasket 19 is placed on the surface of the flange 18, the front edge is pulled in and pressed upon the gasket 19 in order to compress the gasket 19 and form an air-tight seal between the distal end 22 of the hose 20 and the outlet port 12.

A second electrical coupling 70 protrudes from an outer surface 52 of the front lip 57 along a bottom of the front edge 71. The second electrical coupling 70 includes hosing having a plurality of electrical components 72 disposed therein. The plurality of electrical components 72 are in communication with a first wiring 73 extending from the distal end 22 of the hose 20 to the proximal end 21 of the hose 20. The first wiring 73 is further in communication with the control interface 30 at the warming unit coupling 24 at the proximal end 21 of the hose 20. The housing of the second electrical coupling 70 is configured to receive and retain the coupling tab 16 of the first electrical coupling 40 such that the first and second electrical coupling 40, 70 are in electronic communication. The coupling of the first electrical coupling 40 to the second electrical coupling 70 provides electronic communication between the control interface 30 on the proximal end 21 of the hose 20 to the forced air warming device 1 connected to the distal end 22 of the hose 20. Therefore, operational control of the forced air warming device 1 is located near the patient warming unit 2, operational control of the forced air warming unit to the patient or subject wearing the patient warming unit 2.

Referring to FIGS. 4A-6, the cam 50 is rotatable between a first position wherein the tab 16 is positioned within the groove 58 opening. In the first position, the hose 20 is removable from the outlet port 12 of the forced air warming device 1. The cam 50 is then rotatable from a first position to a second position. Rotation of the cam 50 causes the tab 16 of the outer coupling 13 to slide from the groove 58 opening further into the groove 58. The groove 58 may include at least one stop point 80 for receiving and retaining the tab 16 once the front edge 75 of the hose 20 is in contact with the surface of the flange 18 or the gasket 19 and an air tight seal has been formed between the hose 20 and the outer coupling 13. Once the tab 16 is moved into the stop point, the tab 16 is secured within the groove 58, preventing the hose 20 from becoming uncoupled from the forced air warming device 1 outlet port 12.

In some embodiments, the cam may be rotatable between 0 to 25 degrees. In another embodiment the cam may be rotatable between 0 to 25 degrees. In another embodiment the cam may be rotatable between 0 and 30 degrees. In a preferred embodiment, the cam is rotatable between 0 and 35 degrees. When the cam is rotatable fully in the first direction, the tab is retained in the groove and the hose is securely retained to the forced air warming unit and the first and second electrical couplings 40, 70 are in electrical communication. Due to the cam being rotatable around the around the hose the first and second electrical couplings are able to remain stationary while connected, thus reducing the risk of decoupling the couplings and the loss of

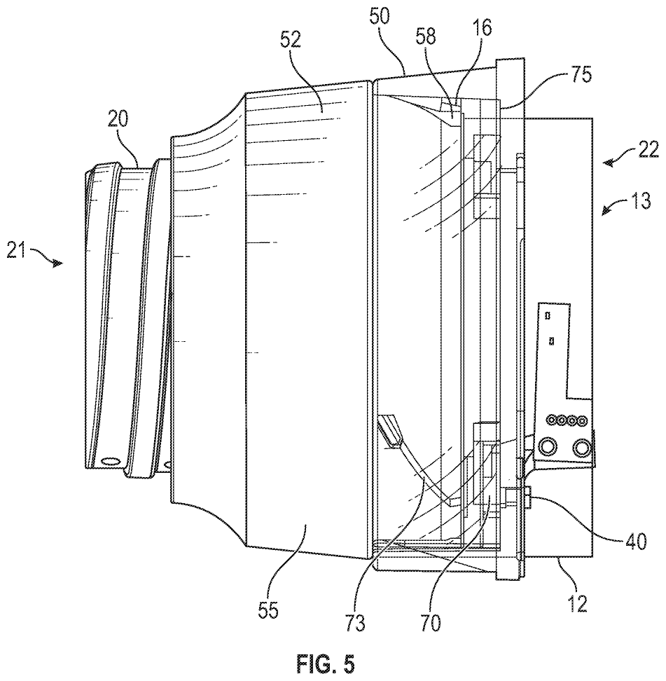

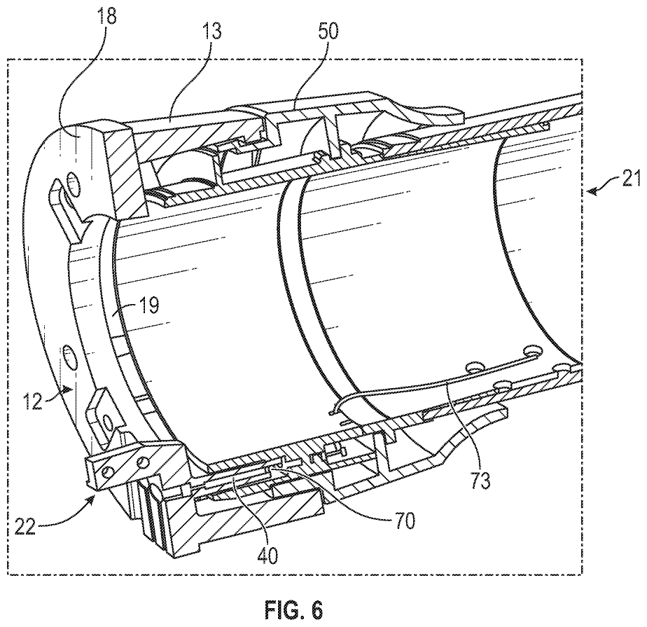

As shown in FIGS. 5 and 6, the coupling of the outlet port 12 of the forced air warming device 1 enables a secure connection between the first and second electrical coupling 70 without having to compromise the electrical communication between the control interface (not shown) and the forced air warming unit. Furthermore, rotation of the cam 50 forces the distal end 22 of the hose 20 into contact with the surface of the flange 18 or the gasket 19 in order to create an air-tight seal. Once the distal end 22 is drawn into the outer coupling 13 the gasket 19 may be compressed in order to ensure that pressurized air may not escape the pathway between the outlet port 12 and the hose 20.

Once the patient warming device may be removed from the forced air warming device 1, rotation of the cam 50 in a second direction releases the tab 16 from the groove 58 and the hose 20 may be easily removed from the outer coupling 13 for cleaning or replacement with a separate patient warming device without compromising the first and second electrical couplings 40, 70.

1. A forced air patient warming system, comprising:
a forced air warming device including a device body;
an air outlet port on the device body, the air outlet port including an outer coupling, the outer coupling comprising:
a wall defining a periphery of the outer coupling, the wall including a tab extending inwardly from an inner surface of the wall;
a first electrical coupling in communication with the device body;
a hose for coupling to the air outlet port, the hose comprising:
a proximal end and a distal end opposite the proximal end;
an opening at the distal end of the hose for receiving forced air from the outlet port;
a first wiring extending from the distal end of the hose to the proximal end of the hose;
a second electrical coupling in communication with the first wiring.
a cam rotationally secured to the hose, the cam comprising:
an outer surface and an inner surface opposite the outer surface;
a groove on the outer surface of the cam for receiving and retaining the tab of the outer wall of the outer coupling, wherein rotation of the cam in a first direction secures the hose to the outer coupling to form a seal between the hose and the air outlet port;
a patient warming device comprising coupled to the proximal end of the hose, the patient warming unit comprising an interface in communication with the first wiring; and
wherein the interface comprises an actuator for controlling operation of the forced air warming device.

2. A forced air patient warming system, comprising:
A forced air warming device including a device body;
an air outlet port on the device body, the air outlet port including an outer coupling, the outer coupling comprising:
a wall defining a periphery of the outer coupling, the wall including a tab extending inwardly from an inner surface of the wall;
a flange extending inwardly from an inner surface of the outer coupling;
a hose for coupling to the air outlet port, the hose comprising:
a proximal end and a distal end opposite the proximal end;
an opening at the distal end of the hose for receiving forced air from the air outlet port;
a cam rotationally secured to the hose, the cam comprising:
an outer surface and an inner surface opposite the outer surface;
a groove on the outer surface for receiving and retaining the tab of the outer wall of the outer coupling;
wherein upon coupling of the hose to the air outlet port, rotation of the cam in a first direction secures the hose to the outer coupling to form a seal between the distal end of the hose and the first surface of the inner flange.

3. The forced air patient warming system of claim 1, further comprising a gasket disposed between the first surface of the inner flange of the outer coupling and the distal end of the hose.

4. The forced air patient warming system of claim 3, wherein the gasket is compressed between the inner flange and distal end of the hose upon rotation of the cam in the first direction.

5. (canceled)

6. A forced air patient warming system of claim 2, wherein the hose comprises a first portion and a second portion, and wherein the first portion is coupled to the second portion to secure a rim of the cam to the hose.

7. The forced air patient warming system of claim 2, wherein the second portion comprises the distal end.

8. The forced air patient warming system of claim 2, wherein:
the outer coupling further comprises a protuberance including a first electrical connector; and
the hose includes a housing and a second electrical connector disposed inside the inner housing.

9. The forced air patient warming system of claim 8, wherein the housing receives the protuberance of the outer coupling when the hose is coupled to the air outlet port, such that the first electrical connector of the outer coupling is in communication with the second electrical connector of the hose.

10. The forced air patient warming system of claim 8, wherein the protuberance extends from the first surface of the inner flange.

11. The forced air patient warming system of claim 2, wherein the cam is rotatable by no more than 20 degrees; no more than 25 degrees; no more than 30 degrees; no more than 35 degrees.

12. The forced air patient warming system of claim 2, wherein the cam is movable between a first position where the hose is removable from the air outlet port and a second position wherein the tab is secured within the grooves of the cam and the hose is secured to the air outlet port.

13. A forced air patient warming system comprising:
a forced air patient warming device comprising:
an air outlet port;
an outer coupling on the air outlet port, the outer coupling comprising;
a wall defining a periphery of the outer coupling, the wall having an inner surface including a tab;
an inwardly extending flange having a first surface and a second surface;
a projection including a first electrical connector extending from the first surface of the flange in a direction parallel to the inner surface of the wall;
a hose for connecting to the forced air patient warming device comprising:
a proximal end and a distal end opposite the proximal end;
an opening at the distal end;
a housing including a second electrical connector for coupling to the first electrical connector;
a cam rotatably secured to an outer surface of the hose comprising a groove for receiving and retaining the tab of the outer coupling; and
wherein the cam rotates to secure the hose to the air outlet port and form an electrical connection between the first electrical connector and the second electrical connector.

14. The system of claim 13, wherein the cam includes an outer surface and an inner surface opposite the inner surface, and wherein the outer surface of the cam includes the groove.

15. (canceled)

16. The system of claim 13, wherein the hose further comprises a first portion and a second portion including the opening at the distal end of the hose, and wherein the first portion is molded to the second portion to secure the cam to the hose.

17. The system of claim 13, wherein the hose includes at least one wire extending from the second electrical connector to the proximal end of the hose.

18. The system of claim 13, wherein the cam is rotatable by no more than 20 degrees; no more than 25 degrees; no more than 30 degrees; no more than 35 degrees.

19. The system of claim 13, wherein the cam is rotatable from a first position where the hose is removable from the air outlet port, and a second position wherein the hose is secured to the air outlet port and the first electrical connection is in communication with the second electrical connection.

20. The system of claim 13 further comprising a gasket located on the first surface of the inwardly extending flange, and optionally on the distal end of the housing.

21. The system of claim 13, wherein the grooves include stop points for securing the tab of the outer coupling within the grooves.

22. The system of claim 13 wherein the distal end of the hose includes a lip for forming an air-tight seal between the distal end of the hose and the first surface of the flange.

23.-29. (canceled)
